(19) United States
(12) Patent Application Publication
(10) Pub. No.: US 2015/0258152 A1
(43) Pub. Date: Sep. 17, 2015

(54) PLANT SPECIES THAT ARE AN EFFECTIVE TREATMENT FOR VIRAL INFECTIONS IN ANIMALS AND PLANTS

(71) Applicant: Warren E. GOODEN, (US)

(72) Inventor: Warren E. Gooden, West Chester, PA (US)

(21) Appl. No.: 14/434,285

(22) PCT Filed: Oct. 10, 2013
(86) PCT No.: PCT/US13/64361

§ 371 Date: Apr. 8, 2015

Related U.S. Application Data
(60) Provisional application No. 61/711,998, filed on Oct. 10, 2012.

(51) Int. Cl. A61K 36/03; A61K 9/00

(52) U.S. Cl. CPC A61K 36/03; A61K 9/0014

(57) ABSTRACT

Methods of making compositions by extracting a plant of the genus Sargassum are described herein. Compositions comprising extracts of a plant of the genus Sargassum are described herein. Methods of treating by applying or administering a composition herein are described. The methods include methods of treating viral infections, methods of removing or preventing the growth of skin moles, and methods of smoothing skin.

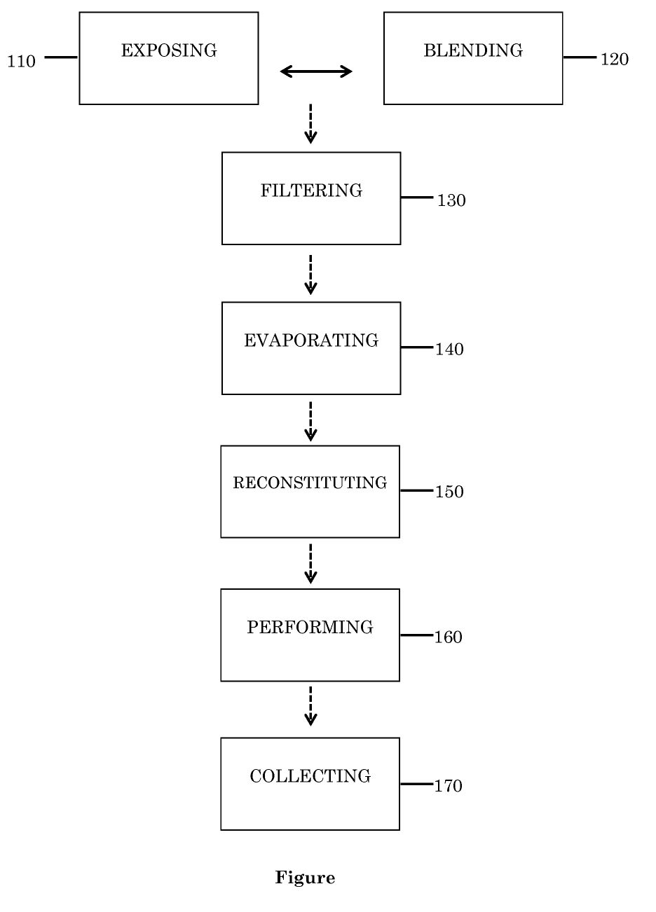

This application claims the benefit of U.S. Provisional Application No. 61/711,998, filed Oct. 10, 2012, which is incorporated herein by reference as if fully set forth.

### FIELD

The disclosure relates to plant extracts with anti-viral activity.

### BACKGROUND

Extracts of some plant species have been shown to exhibit anti-viral activity. Viral infections are common infectious diseases. Common viral infections include infections with herpes simplex virus, poxviruses, or papaviruses.

Poxviruses, members of the family poxviridae, are viruses such as smallpox, vaccinia, cowpox and monkey pox that infect both vertebrate and invertabrate animals.

Papaviruses, members of the former virus family papovaviridae, include human papillovirus, which causes small benign tumors, known as papillomas or warts.

Herpes simplex virus is a prevalent recurrent and chronic infection. Chronic outbreaks of lesions are common in persons infected with herpes simplex virus, occurring usually in mucous membrane areas and the surrounding skin. These lesions occur commonly as orofacial or genital lesions referred to as “cold sores” or “fever blisters,” or sexually transmitted diseases. There is currently no systemic cure for the herpes simplex virus. Shingles is associated with Herpes Zoster.

### SUMMARY

In an aspect, the invention relates to a method of making a composition. The method includes extracting a plant of the genus Sargassum to create an extract of the plant. The composition comprises the extract of the plant.

In an aspect, the invention relates to a method of making a composition. The method includes exposing a plant of the genus Sargassum to a first solvent to obtain a plant-solvent solution; and blending the plant-solvent solution.

In an aspect, the invention relates to a composition comprising an extract of a plant of the genus Sargassum.

In an aspect, the invention relates to a method of treating a patient. The method includes applying or administering a composition comprising an extract of a plant of the genus Sargassum to the patient.

### BRIEF DESCRIPTION OF THE DRAWINGS

The following detailed description of the preferred embodiments of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

The FIGURE illustrates an exemplary method of making a composition.

### DETAILED DESCRIPTION

Certain terminology is used in the following description for convenience only and is not limiting. The words “a” and “one,” as used in the claims and in the corresponding portions of the specification, are defined as including one or more of the referenced items unless specifically stated otherwise. The phrase “at least one” followed by a list of two or more items, such as “A, B, or C,” means any individual one of A, B or C as well as any combination thereof.

Embodiments include methods of making a composition. The method may include extracting a plant of the genus Sargassum to obtain an extract. Sargassum is an aquatic plant capable of asexual and sexual reproduction. Sargassum is in the family Sargassaceae and the order Fucales. The species of Sargassum may be Sargassum muticum. The plant may be an asexually reproducing. As used herein, “plant” may refer to the entire plant, a part thereof, or one or more plant parts. The one or more parts may include but are not limited to the plant holdfast, stipe, or frond. Another plant part or collection of parts may be used to prepare the extracts. The extract may be aqueous or non-aqueous. Extracting may include physical destruction of the plant. For example, extracting may include chopping, slicing, grinding, or smashing the plant. Extracting may include exposing the plant to a first solvent. Extracting may include allowing the plant in the first solvent stand for a period of time. The plant or plant parts may be ground or cut before, during or after being placed in the first solvent.

The first solvent may be an amphipathic solvent. For example, the first solvent may be acetone or an acetone-water mixture. The first solvent may be or include an alcohol, a polar solvent, a protic solvent, or an aprotic solvent. An alcohol as a solvent may include but is not limited to at least one of methanol, ethanol, propanol, or butanol. The alcohol may include a 50:50 ethanol-water mixture or a 50:50 methanol-water mixture. The alcohol may be an ethanol solution. The ethanol solution may include but is not limited to a solution comprising 40% ethanol and 60% H₂O. Extracting may include soaking the Sargassum plant in the first solvent for a period of time. The period of time may be but is not limited to 2 days, 1 day, 12 hours, 8 hours, 6 hours, 3 hours, 2 hours, 1 hour, ½ hour, 15 min., 10 min., 5 min., or 1 min., or a time in a range between any of the foregoing times. Exposing the plant to the first solvent results in a plant-solvent solution.

In an embodiment, the plant-solvent solution obtained from the plant-first solvent mixture may be separated into acidic, basic and neutral fractions. For example, the basic fraction may be separated and used as the extract. Alternatively, the plant or plant parts may be ground or cut and used directly as the extract.

Extracting may include blending the Sargassum plant. Blending may occur prior to exposing the Sargassum plant or part thereof to a solvent. Blending may occur during the step of exposing the Sargassum plant to the first solvent. Blending may be accomplished with a household blender or similar appliance.

Extracting may also include concentrating the plant-solvent solution to form a concentrated plant-solvent solution. Concentrating may be but is not limited to one or more of evaporating or lyophilizing the plant-solvent solution. The concentrated plant-solvent solution may be made into an amphipathic formulation.

The plant-solvent solution may comprise metabolites from within and/or on the plant surface.

Extracting may further include filtering the plant-solvent solution to obtain a filtered solution. Filtering may be accomplished with a Buchner funnel, a vacuum flask, and Whatman filter paper to obtain the filtered solution.

Extracting may include evaporating the filtered solution. Evaporating may be performed in a porcelain or glass evaporating dish. Evaporating may occur at room temperature. Evaporating may occur at 40 degrees Celsius or less. Evaporating may occur under vacuum. Evaporating the filtered solution may result in a residue.

Extracting may include reconstituting the residue in a second solvent. The second solvent may be the same as the first solvent. The second solvent may include an alcohol. The alcohol may include a methanol solution. The methanol solution may comprise 30% methanol, 70% H₂O and 0.1% trifluoroacetic acid. Reconstituting the residue may result in a reconstituted residue. Extracting may include performing High Performance Liquid Chromatographic (HPLC) on the reconstituted residue. Performing HPLC may include filtering the reconstituted residue to obtain a filtered reconstituted residue. Filtering the reconstituted residue may include passing the reconstituted through the filter of a Gelman syringe filter with a Luer lock adapter. Extracting may include injecting the reconstituted residue or filtered reconstituted residue into an HPLC Rheodyne injection system. The pump for the mobile phase may range from 2200 to 2600 psi for a 1 ml per minute flow rate through the HPLC System.

The HPLC system may consist of an HPLC pump, and stainless steel standard HPLC tubing ( 1/16 inch ID) running approximately 6 inches from the pump to the Rheodyne injection system then approximately 3-4 inches to the Agilent Zorbax SB-C18 Column (Column dimensions may be 4.6×250 mm with 5 μm particle size and Serial Number USCL037662). The tubing may be 6 inches in length running from the column to a uv/vis Spectrophotometer containing a flow through cell. A 6 inch length stainless steel tube of the usual bore ( 1/16 inch ID) may deliver the eluent containing plant metabolites into collection tubes.

Extracting may include collecting the mobile phase. The solvent system may be isocratic. Collecting may include collecting continuously every X minutes from the time the sample is injected into the effluent of the HPLC system. X minutes may be 20, 19, 18, 17, 16, 15, 14, 13, 12, 11, 10, 9, 8, 7, 6, 5, 4, 3, 2, or 1 minutes, or a time in a range between any of the foregoing times. X minutes may be 5 minutes. A fraction collected between ten and twenty minutes may be referred to as a first fraction. A fraction collected between thirty-five and forty-five minutes may be referred to as a second fraction. The first fraction includes a composition herein having anti-viral activity. The second fraction, between thirty-five and forty-five minutes, includes a composition herein and has the most potent metabolites eluting from the HPLC column. The most potent metabolites include anti-viral activity.

Referring to the attached FIGURE, an exemplary method 100 of making a composition herein is illustrated. A composition herein may be obtained by collecting the products of after any one step in the flow chart illustrated in the FIGURE. The method may include step 110 of exposing the plant or plant parts to the first solvent, as described above, to obtain a plant-solvent solution; and/or step 120 of blending the plant-solvent solution. In the case that blending 120 occurs in the absence of exposing 110, the resultant mixture from blending may be collected as a composition herein, or a composition herein may be formed by collecting the products of one or more the remaining steps directly. If insufficient liquid exits in the resultant mixture, a third solvent may be added after blending to accomplish exposing 110 and/or facilitate the remaining steps. The third solvent may be but is not limited to being the same as the first solvent. The third solvent may be water. The method may also include step 130 of filtering the plant-solvent solution to obtain a filtered solution. The method may include step 140 of evaporating the filtered solution to obtain a residue. The method may also include step 150 of reconstituting the residue in the second solvent to obtain a reconstituted residue. The method may also include a step 160 of performing HPLC on the reconstituted residue, and step 170 of collecting a fraction from the mobile phase. The method may include collecting at least one of the first fraction or the second fraction as described herein.

Embodiments may include compositions comprising any extract of Sargassum obtained by any of the foregoing methods. The extracts may be included in compositions such as topical skin compositions, edible compositions, injectable compositions, oral compositions and pharmaceutical compositions. Compositions may include any concentration of Sargassum extract. For example, compositions may include a Sargassum extract at a concentration of at least 50 μg per μL. An embodiment includes compositions of a Sargassum extract for internal applications. The extract is effective, and specific compounds involved in the antiviral activity and the mechanism of the interaction between the virus, the mammalian cell infected with the virus, and the compound(s) were and continue to be investigated. An embodiment includes a composition including isolated substances from the extract and having activity. The activity may be anti-viral activity.

Embodiments include topical skin care compositions. Topical skin care compositions may include any composition arrived at by a method herein, or an isolated substance therefrom, and a dermatologically acceptable vehicle. The topical skin care compositions may include a lotion, cream, gel, serum, ointment, paste or emulsion.

A dermatologically acceptable vehicle is a vehicle that is capable of having the plant extract dispersed or dissolved therein, and possesses acceptable safety properties, for example, irritation and sensitization characteristics. The dermatologically acceptable vehicle may be, but is not limited to, a solution carrier system.

Lotions, creams, and ointments can be made from a solution carrier system. In an embodiment, a composition including any composition arrived at by a method herein, or an isolated substance therefrom, further includes a solution carrier system to form a lotion. Lotions typically comprise from about 1% to about 20% of an emollient(s) and from about 50% to about 90% of water. Lotions herein may comprise the emollients and water, and the amounts thereof may be about 1% to about 20% of an emollient(s) and from about 50% to about 90% of water. In an embodiment, a composition including any composition arrived at by a method herein, or an isolated substance therefrom, further includes a solution carrier system to form a cream. A cream typically comprises from about 5% to about 50% of an emollient(s) and from about 45% to about 85% of water. Creams herein may include emollients and water, and the amounts thereof may be 5% to about 50% of an emollient(s) and from about 45% to about 85% of water. In an embodiment, a composition including any composition arrived at by a method herein, or an isolated substance therefrom, further includes a solution carrier system to form an ointment. An ointment herein may comprise a simple base of animal or vegetable oils or semi-solid hydrocarbons (oleaginous, absorbent, emulsion and water soluble ointment bases). An ointment herein may also comprise absorption ointment bases that absorb water to form emulsions. Ointment carriers may also be water-soluble. An ointment herein may comprise from about 2% to about 10% of an emollient(s) plus from about 0.1% to about 2% of a thickening agent(s). Certain ions may be beneficial to the extract's potency, including but not limited to magnesium and calcium, which are known to enhance enzyme activity and protein function, and any composition herein may include ions either from the source or extraction process, or through supplementation of the product with the ion. The ion may be but is not limited to at least one of magnesium ion or calcium ion.

In an embodiment, a composition including any composition arrived at by a method herein, or an isolated substance therefrom, further includes a carrier system and formulated as an emulsion. If the carrier is formulated as an emulsion, typically from about 1% to 10% of the carrier system comprises an emulsifier(s). Emulsifiers may be nonionic, anionic or cationic. Lotions and creams herein may be formulated as emulsions.

Embodiments include methods of treating viral infections by the externally applying of any one or more of the compositions contained herein to the skin of a patient in need thereof. External application of compositions including extracts of the plant genus Sargassum are effective in the treatment of viral infections. Viral infections treated may be but are not limited to infections by herpes simplex virus, poxviruses, and papaviruses. For example, viral infections treated may include but are not limited to herpes simplex II virus, human papilloma virus, herpes zoster, and vaccinia.

A high concentration of the whole plant extract exhibited the most activity. Extract from the plant was administered to the target mammalian cells before and after infection.

Embodiments include methods of treating viral infections by administering any one or more of the compositions contained herein to a patient in need thereof. Administering may be by any route, including but not limited to at least one of topical, oral, intramuscular, intravenous, intra-arterial, anally, or vaginally.

Embodiments include methods of removing or preventing development of skin moles by the externally applying of any one or more of the compositions herein to the skin of a patient in need thereof. External application of compositions including extracts of the plant genus Sargassum may be effective in the removal of common fleshy skin moles (nevi).

Embodiments include methods of smoothing skin by the externally applying of any one or more of the compositions herein to the skin of a patient in need thereof. External application of compositions including extracts of the plant genus Sargassum may be effective in the removal of common fleshy skin moles (nevi).

The patient may be a human patient. The patient may be a nonhuman animal patient. The nonhuman animal may be a mammalian animal. The patient may be a vertebrate. The patient may be an invertebrate animal. The patient may be a plant.

### LIST OF PARTICULAR EMBODIMENTS

The following list includes particular embodiments. The list, however, is not limiting and does not exclude the embodiments otherwise described herein or alternate embodiments.
1. A method of making a composition comprising: extracting a plant of the genus Sargassum to obtain an extract.
2. The method of embodiment 1, wherein extracting comprises exposing the plant to a first solvent to obtain a plant-solvent solution.
3. The method of any one or more of embodiments 1 and 2, wherein extracting further comprises blending the plant-solvent solution.
4. The method of any one or more of embodiments 2-3, wherein the first solvent is an ethanol solution.
5. The method of any one or more of embodiments 2-4, wherein the first solvent is 40% ethanol and 60% H₂O.
6. The method of any one or more of embodiments 2-3, wherein the first solvent is selected from the group consisting of an alcohol, a polar solvent, a protic solvent, and an aprotic solvent.
7. The method of embodiment 6, wherein the alcohol is selected from the group consisting of methanol, ethanol, butanol, and propanol.
8. The method of any one or more of the preceding embodiments further comprising: filtering the product of the one or more embodiments, for example the plant-solvent solution of embodiment 2, to obtain a filtered solution.
9. The method of any one or more of the preceding embodiments further comprising evaporating the product of the one or more embodiments, for example the filtered solution of embodiment 8, to obtain a residue.
10. The method of any one or more of the preceding embodiments further comprising reconstituting the product of the one or more embodiments, for example the residue of embodiment 9, in a second solvent to obtain a reconstituted residue.
11. The method of any one or more of the forgoing embodiments further comprising performing HPLC on the product of the one or more embodiments, for example the reconstituted residue of embodiment 10; and collecting at least one fraction eluting from the HPLC column.
12. The method of embodiment 11, wherein performing HPLC includes implementing an HPLC system comprising a Rheodyne injection system, an Agilent Zorbax SB-C18 Column having dimensions of 4.6×250 mm and a 5 μm particle size, an HPLC pump for a mobile phase, stainless steel 1/16 inch ID HPLC tubing running approximately 6 inches from the HPLC pump to the Rheodyne injection system and approximately 3-4 inches to the Agilent Zorbax SB-C18 Column; and wherein implementing includes injecting the reconstituted residue, optionally filtered reconstituted residue, in the Rheodyne injection system, pumping the mobile phase at 2200 to 2600 psi and a 1 ml per minute flow rate through the HPLC system, and the at least one fraction includes at least one of a first fraction eluting between 10 and 20 minutes and a second fraction eluting between 35 and 45 minutes.
13. The method of any one or more of the preceding embodiments, wherein the plant is Sargassum muticum.
14. A composition comprising an extract of a plant of the genus Sargassum, wherein the extract is obtained by the method of one or more of embodiments 1-13.
15. The composition of embodiment 14 further comprising a dermatologically acceptable vehicle.
16. The composition of one or more of embodiments 14-15 further comprising a carrier system.
17. A method of treating a patient comprising applying the composition of any one or more of embodiments 14-16 to the skin of a patient in need thereof.
18. The method of embodiment 17, wherein the patient is a patient infected with a virus, and applying includes applying the composition to an area of the skin infected with the virus.
19. The method of embodiment 18, wherein the virus is selected from the group consisting of a double stranded DNA virus, Herpes simplex I, Herpes simplex virus II, Vaccinia, smallpox, poxviruses, a papavirus, and Human papilloma virus.
20. The method of embodiment 17, wherein the patient is a patient having or developing moles, and applying includes applying the composition to an area of the skin having or developing the moles.
21. The method of embodiment 17, wherein the patient is a patient desiring smoother skin, and the applying includes applying the composition to an area of the skin the patient desires to be smoothed.
22. A method of treating comprising administering the composition of any one or more of embodiments 14-16 to patient in need thereof.
23. The method of embodiment 22, wherein the patient is a patient infected with a virus.
24. The method of embodiment 23, wherein the virus is selected from the group consisting of a double stranded DNA virus, Herpes simplex I, Herpes simplex virus II, Vaccinia, smallpox, poxviruses, a papavirus, and Human papilloma virus.
25. The method of any one or more of embodiments 22-24, wherein administering is by at least one of the routes selected from the group consisting of topical, oral, intramuscular, intravenous, intra-arterial, anally, and vaginally.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

Substances have been identified in two Sargassum aquatic plants screened. Preliminary studies show that one of the substances may be an alkaloid residing in the basic fraction of plant sample extracts. The compound appears to be labile under basic conditions above pH 9. The other plant sample contains at least one substance that is extractable in either an alcohol or water. These substances may have anti-viral activity.

### Example 2

HSV-2 Confluent rat liver epithelial cells (WB) grown as monolayers in Corning T-25 tissue culture flasks are infected with 1 ml of either HSV-2 or vaccinia virus diluted in medium to yield between 100-250 plaques. A mock-infected negative control is always included. Cells so infected are incubated at 37 degrees C. for one hour to allow the virus to attach to cells. After the hour incubation, unadsorbed virus is washed off with serum-free medium and fed with medium containing the indicated amount of extract. The flasks are incubated for 48 hours. The cells are fixed with 2% formaldehyde in buffer (PBS) for 10 minutes before staining with crystal violet (1% in 35% methanol-PBS). Antiviral activity is measured as a percent reduction in the number of plaque forming units (pfu) compared to the untreated infected controls.

The above assay has been conducted, and the results suggest that a substance existing in or on the product of example 1 is capable of inhibiting Herpes simplex II viral replication and transmission in mammalian cells. The extract used in this experiment was at the microgram per milliliter concentration level. Purification of plant extracts may include solvent extraction followed by chromatographic methods development. Compound structure using mass spectral, Infrared, nuclear magnetic resonance, and x-ray crystalographic analyses could be performed.

### Example 3

When Sargassum muticum was subjected to a 40% ethanol/60% H₂O solution, and allowed to soak for a period of time after blending with a household blender, metabolites from within and on the plant surface were extracted into the solution upon standing. The blended plant and solution were subjected to vacuum filtration using a Buchner funnel with a vacuum flask and Whatman filter paper. The filtered solution was evaporated in a porcelain or glass evaporating dish at room temperature or under vacuum at 40 degrees Celsius or less. The residue was reconstituted in a 30% methanol/70% H₂O/0.1% trifluoroacetic acid solution to be used as a mobile phase in the High Performance Liquid Chromatographic analysis of the extract. A Gelman syringe filter with a Luer lock adapter for the syringe was used for the final filtration prior to injection into an HPLC Rheodyne injection system. The pump for the mobile phase ranged from 2200 to 2600 psi for a 1 ml per minute flow rate through the HPLC System comprising of an HPLC pump, stainless steel standard HPLC tubing ( 1/16 inch ID) running approximately 6 inches from the pump to the Rheodyne injection system then approximately 3-4 inches to the Agilent Zorbax SB-C18 Column (Column dimensions were 4.6×250 mm with 5 μm particle size and Serial Number USCL037662). The tubing was approximately 6 inches in length running from the column to a uv/vis Spectrophotometer containing a flow through cell. A 6 inch length stainless steel tube of the usual bore ( 1/16 inch ID) then delivered the eluent containing plant metabolites into the collection tubes. The mobile phase was collected continuously every 5 minutes from the time the sample was injected into the effluent of the HPLC system. Between ten minutes and twenty minutes, give or take a few minutes, a fraction was collected from the extract that contains metabolites that have an antiviral activity. This fraction was referred to as the “first fraction.” Between 35 to 45 minutes, a fraction began to elute the column. This fraction was referred to as the “second fraction.” The second fraction contains the most potent metabolites to elute the HPLC column. These two fractions are effective against Herpes simplex I and II, Vaccinia, smallpox, poxviruses and other double stranded DNA viruses. This includes Human papilloma virus among other viruses in this family. The mobile phase was isocratic, and the temperature was room temperature.

It is understood, therefore, that this invention is not limited to the particular embodiments disclosed, but is intended to cover all modifications which are within the spirit and scope of the invention as defined by the appended claims; the above description; and/or shown in the attached drawings.

1. A method of making a composition comprising:
extracting a plant of the genus Sargassum to obtain an extract, wherein the composition comprises the extract.

2. The method of claim 1, wherein extracting comprises exposing the plant to a first solvent to obtain a plant-solvent solution.

3. The method of claim 2, wherein extracting further comprises blending the plant-solvent solution.

4. The method of claim 2, wherein the first solvent is an ethanol solution.

5. The method of claim 4, wherein the first solvent is 40% ethanol and 60% H2O.

6. The method of claim 2, wherein the first solvent is selected from the group consisting of an alcohol, a polar solvent, a protic solvent, and an aprotic solvent.

7. The method of claim 6, wherein the alcohol is selected from the group consisting of methanol, ethanol, butanol, and propanol.

8. The method of claim 1 further comprising:
filtering the plant-solvent solution to obtain a filtered solution.

9. The method of claim 8 further comprising evaporating the filtered solution to obtain a residue.

10. The method of claim 9 further comprising reconstituting the residue in a second solvent to obtain a reconstituted residue.

11. The method of claim 10 further comprising performing HPLC on the reconstituted residue;
and collecting at least one fraction eluting from the HPLC column.

12. The method of claim 11, wherein performing HPLC includes implementing an HPLC system comprising a Rheodyne injection system, an Agilent Zorbax SB-C18 Column having dimensions of 4.6×250 mm and a 5 μm particle size, an HPLC pump for a mobile phase, stainless steel 1/16 inch ID HPLC tubing running approximately 6 inches from the HPLC pump to the Rheodyne injection system and approximately 3-4 inches to the Agilent Zorbax SB-C18 Column; and
wherein implementing includes injecting the reconstituted residue, optionally filtered reconstituted residue, in the Rheodyne injection system, pumping the mobile phase at 2200 to 2600 psi and a 1 ml per minute flow rate through the HPLC system, and the at least one fraction includes at least one of a first fraction eluting between 10 and 20 minutes and a second fraction eluting between 35 and 45 minutes.

13. The method of claim 1, wherein the plant is Sargassum muticum.

14. A composition comprising an extract of a plant of the genus Sargassum, wherein the extract is obtained by the method of claim 1.

15. The composition of claim 14 further comprising a dermatologically acceptable vehicle.

16. The composition of claim 14 further comprising a carrier system.

17. A method of treating a patient comprising applying the composition of claim 14 to the skin of a patient in need thereof.

18. The method of claim 17, wherein the patient is a patient infected with a virus, and applying includes applying the composition to an area of the skin infected with the virus.

19. The method of claim 18, wherein the virus is selected from the group consisting of a double stranded DNA virus, Herpes simplex I, Herpes simplex virus II, Vaccinia, smallpox, poxviruses, a papavirus, and Human papilloma virus.

20. The method of claim 17, wherein the patient is a patient having or developing moles, and applying includes applying the composition to an area of the skin having or developing the moles.

21. The method of claim 17, wherein the patient is a patient desiring smoother skin, and the applying includes applying the composition to an area of the skin the patient desires to be smoothed.

22. A method of treating comprising administering the composition of claim 14 to patient in need thereof.

23. The method of claim 22, wherein the patient is a patient infected with a virus.

24. The method of claim 23, wherein the virus is selected from the group consisting of a double stranded DNA virus, Herpes simplex I, Herpes simplex virus II, Vaccinia, smallpox, poxviruses, a papavirus, and Human papilloma virus.

25. The method of claim 22, wherein administering is by at least one of the routes selected from the group consisting of topical, oral, intramuscular, intravenous, intra-arterial, anally, and vaginally.
